(19) United States
(12) Patent Application Publication
(10) Pub. No.: US 2006/0009820 A1
(43) Pub. Date: Jan. 12, 2006

(54) Apparatus for the application of electrical pulses to the human body

(76) Inventor: John Royle, Clitheroe (GB)

Correspondence Address: ADAMS EVANS P.A., 2180 TWO WACHOVIA CENTER, CHARLOTTE, NC, 28282, US

(21) Appl. No.: 10/521,185

(22) PCT Filed: Jul. 16, 2003
(86) PCT No.: PCT/GB03/03235

§ 371 Date: Jun. 24, 2005

(30) Foreign Application Priority Data
Jul. 17, 2002 (GB) 0216567.8
Sep. 5, 2002 (GB) 0220644.9
Dec. 2, 2002 (GB) 0228027.9

(51) Int. Cl. A61N 1/18; A61N 1/30

(52) U.S. Cl. 607/74; 607/3; 604/20

(57) ABSTRACT

An apparatus is described for applying electrical pulses to a patients body by at least two electrodes at respective locations on the patients body, the apparatus comprising a pulse generating unit connectable to the electrodes, the pulse generating unit being arranged to provide a series of electrical pulses, wherein said series of pulses comprises a plurality of first and second polarity impulses having a temporal spacing between the first and second impulses, wherein each impulse has a width of between 2 to 30 μS.

### FIELD OF THE INVENTION

This invention relates to apparatus and methods suitable for, but not limited to, the application of electricity to the skin so as to modulate nerves electronically.

### BACKGROUND OF THE INVENTION

Today, the therapeutic and diagnostic uses of electricity in medicine are widespread. Extensive literature exists on electro-therapy, the therapeutic application of electricity, which is suitable for treatment of a range of medical conditions.

TENS (Trancutaneous Electrical Nerve Stimulation) is the application of electrical pulses via electrodes placed on the skin of a patient, so as to produce a rather short-lived, localised region of analgesia. TENS devices typically utilise pulses of width 50-500 μs, at a current of amplitude 0-50 mA, delivered at a frequency of 80-100 Hz. The TENS pulse is intended to be sufficiently long in duration to excite nerve fibres in the immediate vicinity of the electrodes to cause a painless tingling at low voltage (the voltage amplitude of TENS pulses that can be tolerated by a patient tends to be limited by the level of tingling sensation that can be comfortably endured).

TSE (Trancutaneous Spinal Electroanalgesia) improves upon TENS by providing a longer-lasting form of analgesia, that is more generalised (i.e. not limited to the immediate vicinity of the electrical stimulation). TSE is, for instance, described within U.S. Pat. No. 5,776,170 which describes the original research performed in relation to this treatment.

U.S. Pat. No. 5,776,170 describes how, by applying a continuous series of electrical rectangular pulses to two electrodes, analgesic effects are induced in the central nervous system. The pulses can be a monopolar or bipolar pulse series. The pulses used by the TSE stimulator are typically of 180 volts amplitude (compared with 35-50 volts of the TENS device), with a relatively narrow pulse width (1-10 μs), at frequencies of typically 600-800 Hz.

FIG. 1 illustrates such a continuous bipolar pulse stream. Rectangular pulses 10, 12, 14 of width W, and amplitude Vₚ are delivered at regular predetermined intervals T. The pulse frequency is thus 1/T Hz (when T is expressed in seconds).

In traditional electro-therapy, the efficacy of treatments is generally proportional to the voltage used. However, high voltages are normally both painful to the body, and damaging to tissues. As many electrotherapy devices are powered by batteries, the high energy usage associated with high voltages is also problematic.

Clinical efficacy is also a function of the frequency at which the pulses are delivered. However, whilst the body tissues are typically unharmed by the application of high frequency pulses, the heat generated in the electrodes utilised to apply the pulses can burn the tissues of the body. For instance, U.S. Pat. No. 5,776,170 describes how voltage has to be decreased at high frequencies so as to reduce unwanted heating effects e.g. pulses of amplitude 150 volts can be utilised at a frequency of 5 kHz, whilst the voltage has to be reduced to 25 volts at 150 kHz.

It is an aim of embodiments of the present invention to overcome, or at least alleviate, one or more problems of the prior art, whether referred to herein or otherwise.

### STATEMENTS OF THE INVENTION

In a first aspect of the present invention, there is provided an apparatus for applying electrical pulses to a patients body by at least two electrodes at respective locations on the patients body, the apparatus comprising a pulse generating unit connectable to the electrodes, the pulse generating unit being arranged to provide a series of electrical pulses, wherein said series of pulses comprises a plurality of first and second polarity impulses having a temporal spacing between the first and second impulses, wherein each impulse has a width of between 2 to 30 μS.

Preferably, the first polarity is positive and the second polarity is negative.

Preferably, the first polarity is negative and the second polarity is positive.

Preferably, each impulse has a width of more than 10 μS.

Preferably, each impulse has width of 15 to 20 μS.

Preferably, said series of pulses has a spacing of at least 4 μS between impulses.

Preferably, said series of pulses has a spacing of at least 6 μS between impulses.

Preferably, said series of pulses has a spacing of at least 10 μS between impulses.

Preferably, said series of pulses has a spacing of at least 20 μS between impulses.

Preferably, the series has a maximum spacing of 10 μS between impulses.

Preferably, the apparatus has a maximum spacing of 20 μS between impulses.

A 14 μs gap is thought to be usable in most applications.

Preferably, a temporal space exists between a plurality of contiguous impulses.

Preferably, a temporal space exists between a majority of impulses.

Preferably, a temporal space exists between all impulses.

Preferably, each impulse has an asymmetric shape.

Preferably, the transition time from 0 Volts to a peak magnitude is less than or equal to 30% of the impulse width.

Preferably, the transition time from 0 Volts to the peak magnitude is less than or equal to 10% of the impulse width.

Preferably, the transition time from 0 to the peak magnitude is less than or equal to 5% of the impulse width.

Preferably, the transition time from 0 to the peak magnitude is less than or equal to 1% of the impulse width.

Preferably, the transition time between the positive voltage peak and the negative voltage peak is at least 70% of the pulse period.

Preferably, said impulses have a peak amplitude lying within the range 50 to 450 Volts, plus or minus respectively.

Preferably, each impulse has an amplitude within the range 150 to 250 Volts, plus or minus respectively.

Preferably, the magnitude of positive peak amplitude is substantially equal to the magnitude of the negative peak amplitude.

Preferably, during the spacing between impulses the output of the pulse generating unit remains at a level substantially equal to zero Volts.

Preferably, the series of impulses are delivered at a predetermined frequency lying within the range 100 Hz to 250 kHz. This may be 1 kHz to 5 kHz or more preferably, 2 kHz to 3 kHz.

Preferably, the series of impulses are delivered at a predetermined frequency lying within the range 50 kHz to 250 kHz.

Preferably, the series of impulses are delivered at a predetermined frequency lying within the range 50 kHz to 250kHz.

Preferably, the series of impulses is an intermittent series of pulses.

Preferably, in said intermittent series of electrical impulses, the ratio of the time period for which no impulses are being provided to the time period for which impulses are being regularly provided is within the range 1:3 to 1:20.

Preferably, said ratio is approximately 1:10.

Preferably, at least one pause occurs in said intermittent series of impulses at least once every second

Preferably, said pause is of duration of at least 0.5 millisecond.

Preferably, the apparatus further comprises a battery for providing power to said generating unit for the generation of said pulses.

Preferably, the apparatus further comprises at least two electrodes arranged for connection to said generating unit, for supplying electrical pulses to respective locations on the patients body.

Preferably, the apparatus is for providing therapy to a patient.

Preferably, said apparatus is for supplying electrical pulses to two or more locations on the patients body overlying the central nervous system, such that the pulses induce analgesic effects in the central nervous system, whilst stimulating peripheral nerves that lie between the electrodes and the central nervous system to a lesser extent or not at all.

Preferably, said apparatus is for providing iontophoresis to a patients body by at least two iontophoresis electrodes at respective locations on the patient's body, the apparatus comprising a pulse generating unit connectable to the electrodes, the pulse generating unit being arranged to provide a series of electrical pulses having a peak amplitude of at least 50 Volts.

Preferably, the apparatus further comprises at least two iontophoresis electrodes arranged for connection to said generating unit, for supplying electrical pulses to respective locations on the patient's body, at least one of said electrodes incorporating a medication in ionic form for application to the patient's body.

According to another aspect of the present invention, there is provided a method for applying electrical pulses to a patients body by utilising at least two electrodes at respective locations on the patients body, the method comprising applying an intermittent series of electrical pulses.

According to another aspect of the present invention, there is provided a method for providing iontophoresis to a patient by utilising at least two electrodes at respective locations on the patients body, at least one of the electrodes incorporating an ionic medication, the method comprising applying a series of pulses, each pulse having a peak amplitude of at least 50 Volts to the electrodes, such that the medication is passed into the body of the patient.

### BRIEF DESCRIPTION OF THE DRAWINGS

For a better understanding of the invention, and to show how embodiments of the same may be carried into effect, reference will now be made, by way of example, to the accompanying diagrammatic drawings in which:

FIG. 1 illustrates a typical bipolar pulse train of a known TSE device;

FIG. 2 illustrates a series of impulses in accordance with a first embodiment of the present invention;

FIG. 3 illustrates a series of intermittent impulses according to the present invention.

FIG. 4 illustrates an impulse shape in accordance with the present invention;

FIG. 5 illustrates a second impulse shape in accordance with the present invention;

FIG. 6 is a device suitable for iontophoresis using the spaced impulses according to the present invention;

FIG. 7 is a schematic diagram of a device suitable for producing pulses in accordance with an embodiment of the present invention; and

FIG. 8 illustrates the waveforms at various points in the device shown in FIG. 7.

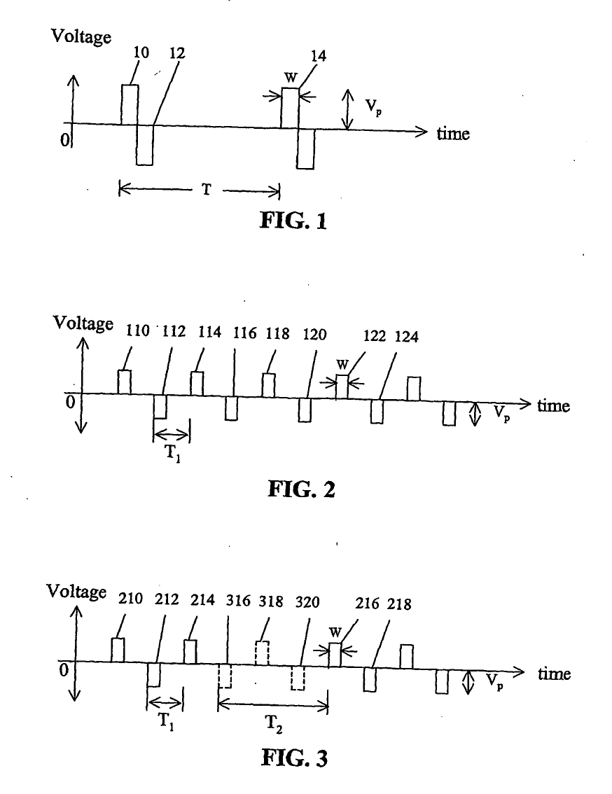

### DETAILED DESCRIPTION OF PREFERRED EMBODIMENTS

The present inventor has realised that, by appropriately changing the waveform applied to the patient, there can be an improvement in the performance of the electrical treatment. This can be achieved by providing positive and negative impulses with a spacing between impulses and optionally the series of pulses may be changed to an intermittent series.

In a first aspect, a series of positive and negative impulses having a spacing T are used instead of the bipolar voltage pulses proposed by the prior art. In initial trials it has been found that a spacing T between impulses proves effective. A spacing of 4 μS or even 6 μS is preferable between impulses. Such impulses are shown in FIG. 2.

Surprisingly, use of such a pulse sequence allows relatively long duration impulses of at least 2 μS and up to 30 μS of relatively high voltage amplitude to be applied to a patient. This enables an increased quantity of electrical charge to be applied to the patient without unwanted side effects, thus increasing the efficacy of the treatment. The spacing provided between positive and negative impulses allows nerve fibres to recover between impulses, enabling improved performance.

Use of both positive and negative voltage impulses has been termed ENM (Electronic Nerve Modulation), and evidence suggests it provides superior treatment to TSE. For instance, ENM appears to alleviate the symptoms of viral infection, and also to decrease the period of infection. It has also been noted that ENM appears to be beneficial in the treatment of patients suffering from epilepsy.

In an optional modification, by providing an intermittent series of impulses, rather than the continuous series of pulses utilised by the prior art, high frequency electrical signals can be applied to a patient without a significant build up of heat in the electrodes. Thus, by using an intermittent series of electrical impulses, then for a given impulse frequency, higher voltages can be utilised without the electrodes burning the skin of the patient. Alternatively, for a predetermined impulse voltage amplitude, higher frequencies can be achieved without damaging tissues.

Further, as the number of impulses delivered in any given interval is reduced compared with a continuous series of impulses, then for a given pulse shape, amplitude and frequency, it will be appreciated that the power required is reduced. Thus, there is an improvement in battery life.

Initial trials have indicated that, despite the number of impulses being reduced due to the intermittent nature of the pulse series, clinical efficacy is not decreased compared with a similar continuous pulse series.

FIG. 3 illustrates an intermittent series of impulses. The series in this example comprises a number of substantially uniformally sized and shaped impulses 210, 212, 214, 216, 218. The impulses are each of width W, with the spacing between each impulse in the series being normally T₁. The impulses have an amplitude of Vₚ volts plus or minus respectively, and in this instance are substantially rectangular in shape. The intermittent series is achieved by providing a pause of temporal duration T₂, during which there are no impulses in the sequence. Preferably, the pause is an integral number of the impulse repeat period T₁ (i.e. T₂=n×T₁, where n is any integer). FIG. 2 illustrates the case where T₂=3T₁, with the 3 dashed impulse shapes 316, 318, 320 indicating those impulses that have effectively been removed from the pulse sequence by the presence of the pause.

In order for nerve modulation to take place, it is desirable that the width W of the impulses lies within the range 2-30 μs. In some instances, the impulse shape may limit the width W. For instance, a patient will normally experience a sensation if a square wave impulse wider than 10 μs is utilised. Other, preferred waveforms are described below that allow longer width impulses to be utilised. Preferably, the impulses have a peak amplitude (Vₚ) within the range of 50 to 450 volts, plus or minus respectively. Preferably, the impulses are delivered at a predetermined frequency (i.e. 1/T₁) lying within the range 100 Hz to 250 kHz. For most applications 2 kH-3 kHz will be used and for medical uses 10 kHz may be the upper frequency limit. The intermittent series of pulses effectively comprises blocks of impulses delivered at the predetermined frequency (1/T₁), with the blocks separated by pauses of duration T₂.

It will be appreciated that the repeat frequency of the pauses can be varied, however it is preferable that the total time period for the pause (i.e. the time period for which no pulse is being provided) compared with the average block length of the pulses (i.e. the time period for which pulses are being regularly provided) lies within the range 1:3 to 1:20. Preferably, the pauses are of duration of at least 1 millisecond (i.e. T₂=1 ms).

Experiments have indicated that an intermittent pause timed at 1.3 milliseconds has no effect on clinical efficacy, but it is anticipated that pauses for longer duration will also be effective, and have either no, or comparatively little effect upon the clinical efficacy. It will be appreciated that in a signal of 2500 Hz, a pause of 1.3 ms is over three times the length of the electronic pulse cycle (i.e. the pulse repeat time, T₁), whilst in a signal operating at 20000 Hz, it is over 25 times the length of the electronic pulse cycle.

It will be appreciated that the maximum frequency can be linked to the wavelength used.

Whilst in the above embodiment, rectangular shaped impulses have been illustrated, it has been discovered that spiked impulses (i.e. pulses with very little signal duration at maximum amplitude) are particularly effective. Such impulses preferably also have relatively fast rise and fall times. This results in the pulse width W being relatively short compared to the length of the pulse cycle (e.g. W is less than 20% of T₁, or more preferably W is less than 10% of T₁, or even less than 5% or 1% of T₁). Spiked impulses are believed to be particularly efficient, as they allow relatively high voltages to be utilised for a given impulse power compared with a rectangular shaped impulse.

Such a series of spaced positive and negative voltage impulses can be used as part of an intermittent series of pulses. Alternatively, the pulses can be used in a continuous series of pulses. Use of either pulse series allows a larger electrical charge to be provided to the patient than suggested by the prior art. For instance, pulses have been used with an amplitude within the range of 100 to 400 volts, without any sensations being experienced by the patient.

The prior art suggests that use of such high voltage pulses would lead to burning within the skin of a patient. However, it is believed that use of both positive and negative voltage pulses prevents the build up of charge within the skin, and hence the skin is less likely to burn.

The use of a fast rise time (the transition time from 0 volts to the peak voltage) of the pulses is preferable, as it is understood to lower the electrical resistance of the skin without stimulating the peripheral nerves, so that the subject (i.e. patient) feels no sensation. Further, this enables a relatively large quantity of electrical charge to pass through the skin and tissues.

It is also preferable that the voltage decays from the respective positive or negative peak voltage to zero volts, so as to ensure that the peripheral nerves are not stimulated.

Preferably, this decay occurs over a relatively long time period (e.g. up to 30 μs), so as to maximise the electrical charge being passed to the patient.

The efficacy of the treatment appears to be related to the pulse width, with wider pulses providing more effective treatment, presumably due to the increase in the total electrical power that can be applied to the patient. By utilising positive and negative voltage impulses as described above, the impulse width can be increased dramatically compared with the impulse width of a rectangular pulse. For instance, typical known rectangular impulses are limited to a width of about 4 μs, as longer rectangular impulses lead to a tingling feeling within the patient. However, using positive and negative voltage impulses, longer pulse widths can be comfortably utilised on a patient e.g. pulses of widths of up to 30 μs, although preferably within the range 10 to 20 μs, and more preferably of a width of substantially 15 μs. This very significant discovery allows a greatly increased electrical charge to be applied to a patient, enabling a range of therapies to be provided for the patient.

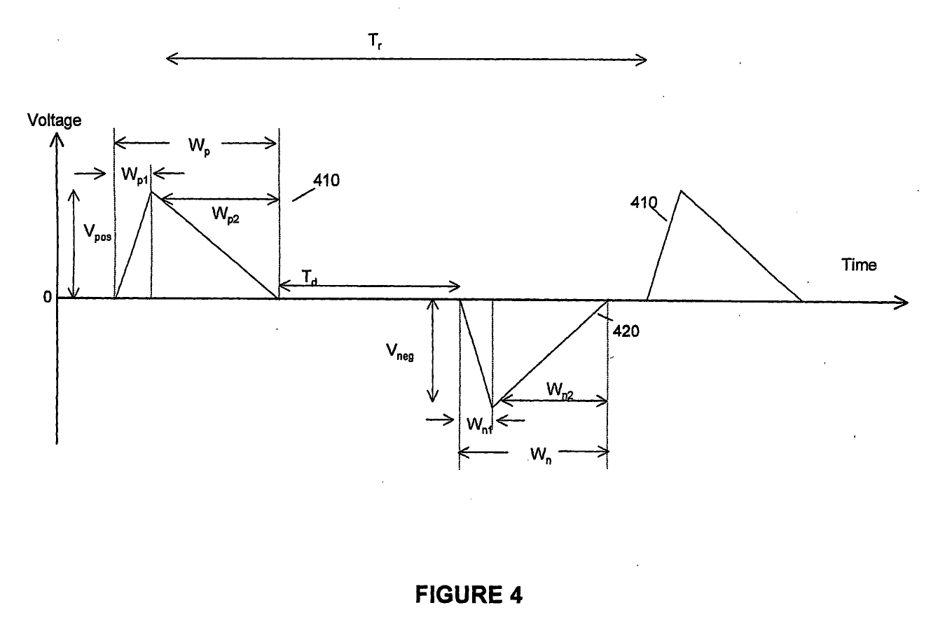

FIG. 4 illustrates a portion of a sequence of alternating asymmetric positive and negative voltage impulses having a spacing between impulses. The positive voltage impulses 410 can be seen to be characterised by a rise time (Wₚ₁), the time taken by the impulse to transition from zero volts to the peak voltage (Vₚₒₛ). In this example, the impulse then immediately decays from the peak voltage Vₚₒₛ back to zero volts, taking a time (Wₚ₂) to return to zero from the peak voltage. The approximate total width of the positive impulse Wₚ is thus: Wₚ=Wₚ₁+Wₚ₂.

After a time delay of T_{d} after the positive voltage impulse, a negative voltage pulse 420 is delivered. The negative voltage impulse takes a time Wₙ₁ to “rise” from zero volts to the peak negative voltage (V_{neg}) and subsequently takes a time Wₙ₂ to fall from the peak negative voltage back to zero volts. Consequently, as the transition from the rising edge of the impulse to the falling edge of the pulse is almost instantaneous, the approximate total width of the pulse Wₙ is Wₙ=Wₙ₁+Wₙ₂.

In this example, positive and negative voltage impulses are alternated, with the repeat period (e.g. the time period between the start of successive positive voltage impulses) being Tᵣ. This repeat period defines the effective frequency of the resulting pulse series (i.e. frequency=1/Tᵣ). It can be noted that the pulse series shown in FIG. 4 has a peak-to-peak duration of over 70% of Tᵣ. Whilst in this example Td is greater than the delay between the negative impulse and the second positive impulse, it will be appreciated that this is merely optional and Td can equal the delay between second and third impulses.

It will be appreciated that the various parameters of these impulses can vary as disclosed generally within this specification. In this example, both the positive and negative voltage impulses are of similar shape, and of similar amplitude and duration. However, any of these parameters of these pulses can be altered. Equally, whilst a delay T_{d} between the pulses is shown to be 6 μS, this delay can in fact take any value from 6 μS up to approximately 1,500 μs. Typically, it is envisaged that each pulse will be of total width of up to 30 μs (i.e. Wₙ≦30 μs, Wp≦30 μs), with the peak voltages of each pulse being within the range 50-450 volts. In trials, such pulses appear to have a strong relaxation effect upon patients.

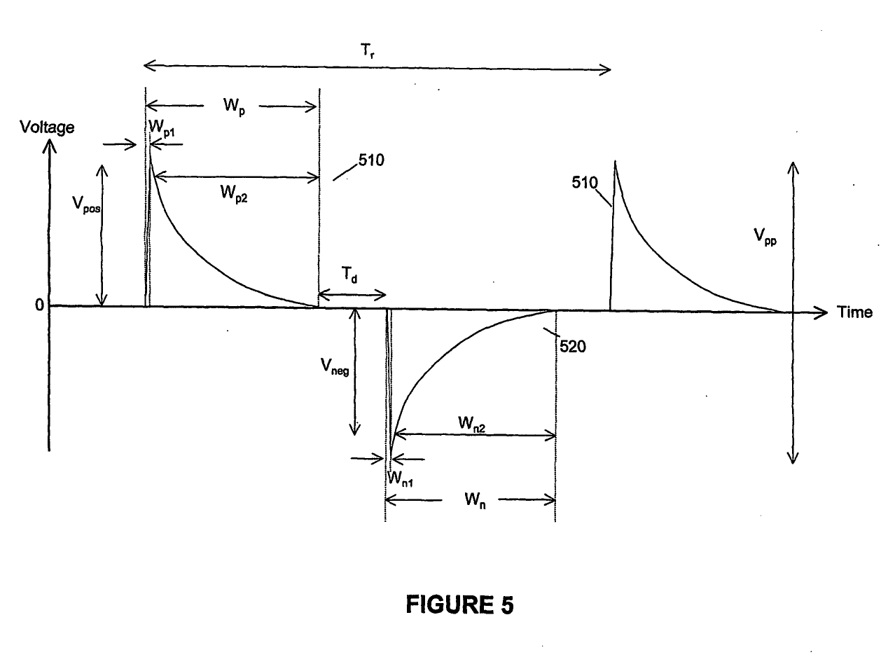

FIG. 5 illustrates an additional impulse shape of the present invention, with in this instance the first peak in the pulses being the positive voltage peak. The pulse cycle is again of length T₁, with the overall pulse width being W. The peak to peak voltage is shown as Vₚₚ, with in this instance both the positive and the negative peaks being of similar amplitude (i.e. half of Vₚₚ). It will be seen that both the positive and negative impulses can be characterised by two time periods (W₁, W₂), where W=W₁+W₂. The initial transition from zero volts to the first peak voltage (in this case, the rise time of impulse) is of duration W₁, the transition time from the first pulse peak to zero volts is of duration W₂.

It is desirable that W₁ is relatively quick compared with the overall pulse width W i.e. W₁≦0.3 W, and more preferably W₁≦0.05 W or W₁≦0.01 W. Preferably, the first differential of voltage change constantly changes during the transition time W₂, and preferably the voltage changes at an exponential rate.

Thus the waveforms provide a contiguous series of impulses with temporal spacings therebetween. The repeat of a first impulse can be regarded as a third impulse with a temporal spacing between the second and third impulses as well as between the first and second impulses.

Iontophoresis is a process which allows for enhanced transdermal drug delivery by use of an applied current through the skin. The application of an electric current causes the migration of drugs or medications, in their ionic form, into the tissues, the migration being proportional to the electrical charge applied through the iontophoretic system. Work on iontophoresis has indicated that applying a voltage to the skin acts to lower the electrical resistance of the skin, the decrease in electrical resistance being proportional to the applied voltage.

A typical apparatus for providing iontophoresis comprises a current source connected to at least two electrodes. The electrodes may be incorporated within a single unit, commonly called a transdermal patch. Typically, one of the electrodes will contain an ionic medication (D⁺ A⁻), and the other an electrolyte (H⁺ A⁻). During iontophoresis treatment, the transdermal patch is applied to the patient's skin and the ionic medication is delivered to the patient with aid of the applied electric current.

The article by Mark R. Prausnitz, “The effects of electric current applied to the skin: A review for transdermal drug delivery”, Advanced Drug Delivery Reviews 18 (1996) 395-425, provides an overview of the effects of electrical current applied to skin. The article describes how, at high voltages, the resistivity of the skin may change rapidly. Electrical burns can result if the electric current flowing through tissues or bones is too high. Burns are believed to be due to the highly localised heating by large current densities at sites of low electrical resistance.

In order to prevent such electrical burns, iontophoresis devices utilise a current source 20 to provide a continuous predetermined level of current (e.g. 2 mA). This typically corresponds to an applied voltage of around 2 Volts, and is understood to rarely exceed 10 Volts.

The present inventor has determined that problems of prior art iontophoresis devices can be overcome by providing iontophoresis using the spaced positive and negative impulses of the present invention. This allows relatively high voltages to be utilised, without any associated burns or sensations. As the current into the body is non-linear with respect to voltage, this allows a proportionally greater current to be utilised, and subsequently a larger amount of medication to be delivered.

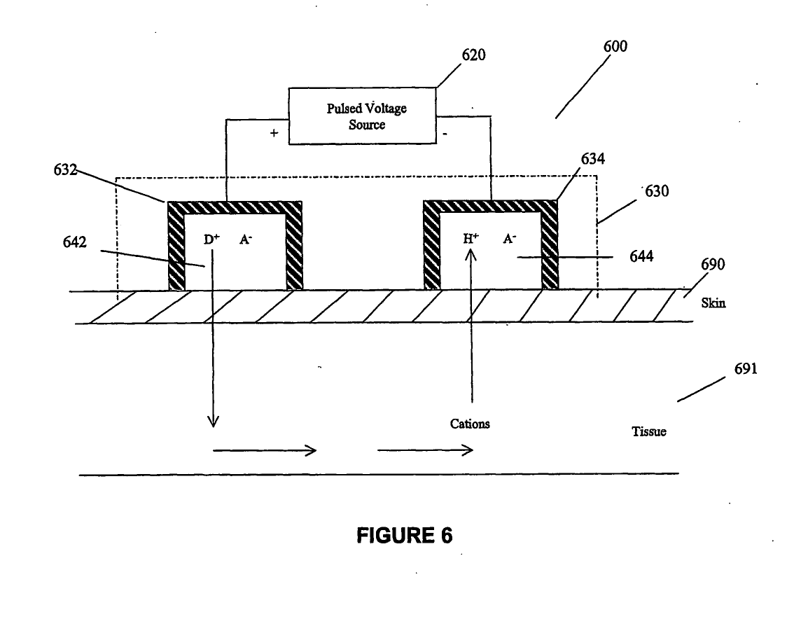

FIG. 6 illustrates an iontophoresis device 600 in accordance with a preferred embodiment of the present invention. The device is powered by a battery (not shown). In use, the electrodes 632, 634 are positioned on the skin 690 of a patient. When a voltage is applied to the electrodes, a circuit is formed between the two electrodes via the body of the patient. The resulting current flowing through the skin 690 of the patient drives the ionic medication into the skin 690 and the tissue 691 to be absorbed by the patients body.

The apparatus is essentially the same as a prior art iontophoresis device, apart from the fact that instead of a DC current source, a pulsed voltage source 620 is utilised to provide a series of spaced positive and negative voltage impulses according to the present invention to the electrodes 632, 634.

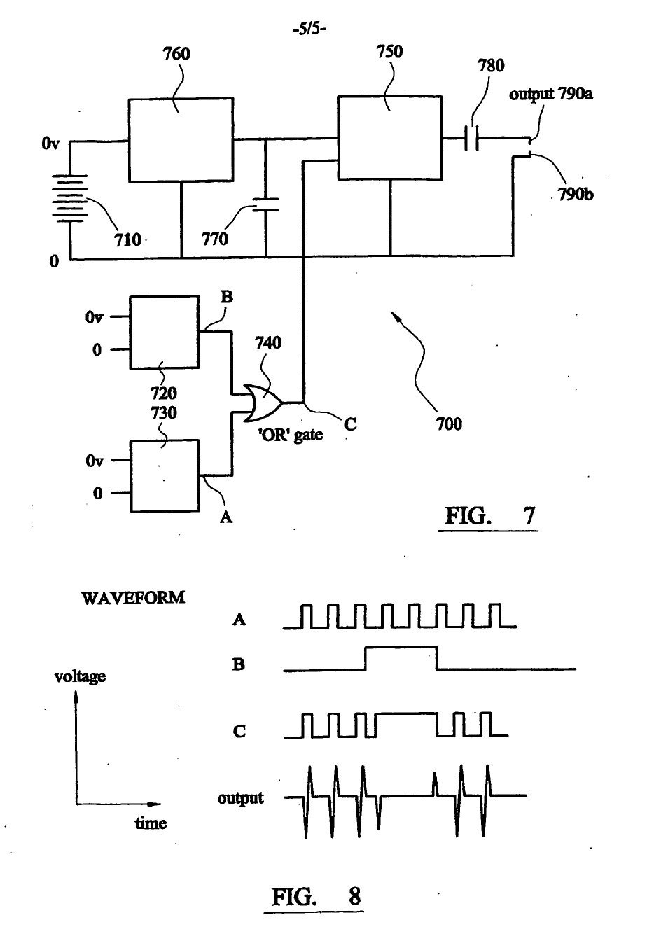

FIG. 7 illustrates an apparatus 700 suitable to automatically produce an intermittent series of alternating positive and negative voltage pulses. The apparatus is powered by a battery 710, supplying a predetermined voltage of “a” Volts.

The apparatus can be envisaged as being in four distinct portions: a continuous fast pulse generator 730; a modulation waveform generator 720, 740; the output pulse shaping unit (760, 770, 750, 780); and the output electrodes 790a, 790b.

FIG. 8 illustrates the waveforms at points marked A, B, C and “output” in the apparatus schematically shown in FIG. 7.

The continuous fast pulse generator 730 is arranged to generate a continuous sequence of impulses at the desired, predetermined positive impulse output pulse frequency. In this instance, the output waveform is of similar shape to that illustrated in FIG. 4, but with a negative first pulse. The waveform A is provided at one input to an OR logic gate 740.

The modulation waveform generator 720 is used to generate a waveform suitable for amplitude modulating the continuous fast pulse generator output, so as to obtain the desired pauses in the pulse series. In this instance, due to the particular implementation of the apparatus, the output of the modulation waveform generator is in fact the inverse of the desired amplitude modulation envelope. Consequently, the waveform B output by the modulation waveform generator 720 is at logic 1 during the desired pause interval (i.e. indicated by T₂ in FIG. 2), and at logic 0 for the remainder of the time.

The OR gate 740 combines the two input waveforms A, B using the logical OR operation, and outputs waveform C.

The high voltage switch 750 is operated by the output of the OR gate 740 i.e. by waveform C. The high voltage switch 750 controls the charging and discharging of capacitor 770.

The capacitor 770 charges up via the operation of a transformer (step up converter) 760, which acts to step up the voltage from the battery power supply 710.

The high voltage switch 750 operates so as to allow the capacitor 770 to be charged up to a relatively high voltage (i.e. approximately the desired peak voltage of the output pulse), with the capacitor being subsequently discharged to the output electrodes 790a, 790b. This output voltage discharge can occur through capacitor 780, which can act to differentiate the signal resulting from the discharge of capacitor 770, and so obtain the desired waveform i.e. an intermittent series of spaced alternating positive and negative voltage impulses.

The output voltage waveform is provided across electrodes 790a and 790b, before application to the body of the patient.

It will be appreciated that the apparatus shown in FIG. 7 can be adapted to generate a continuous series of spaced positive and negative voltage impulses. Such a continuous pulse series generator is achieved by providing the output of the continuous fast pulse generator (A) directly to the input (C) of the high voltage switch 750. In other words, simply deleting the modulation waveform generator 720 and the OR gate 740 from the apparatus results in the apparatus being suitable for providing a continuous series of positive and negative impulses. If desirable, a switching arrangement could be implemented, so as to modify the apparatus shown in FIG. 4 to be used for producing both an intermittent series and a continuous series of impulses. In the first configuration, the connections are shown as in FIG. 5. In the second, switched configuration, output A of generator 730 is connected directly to input C of high voltage switch 750, with the output from the OR gate 740 disconnected from the circuit.

In use, in order to obtain ENM, the electrodes are normally applied to the surface of a body overlying the central nervous system, such that analgesic effects tend to be effected in the central nervous system whilst stimulating peripheral nerves that lie between the electrodes and the central nervous system to a lesser extent or not at all. If desired, the electrodes could be implanted within the body, including within the skin, but it is more preferable that they are designed to simply be placed in contact with the skin surface. Typically, the electrodes are spaced apart by a distance of around 10 cm, and are always over the central nervous system, irrespective of the location of the pain.

In the context of this invention, the term “central nervous system” should be interpreted to include the brain and the spinal cord, and also include the other neural tissues which may otherwise be classed as part of the peripheral nervous system, but are in close anatomical proximity to the central nervous system, such as the ganglia, autonomic or somatic, such as the dorsal root ganglia.

It will be appreciated that the above description is provided by way of example only, and that various other waveforms, and apparatus suitable for producing such waveforms, would be understood as falling within the scope of the present invention. Further, whilst the apparatus has been described in terms of being utilised for ENM, it will be appreciated that other, similar apparatus can make use of the present invention. Electrodes of such apparatus need not be located over the central nervous system when in use.

For instance, evidence suggests that locating the electrodes of a pulse generator on either side of the carotid bodies of a patient can assist in management of the cardio vascular system. Applying this type of pulse as described herein at an operating frequency of approximately 20 kHz, with a peak to peak voltage of between 250-300 volts has been shown to effect cardiovascular system, including altering the pulse rate of a patient.

Further, evidence suggests that application of this type of pulse to patients who suffer from epilepsy appears to reduce the number of epileptic fits.

A double blind placebo controlled crossover trial using ENM technology has just been completed. Although full statistical analyses of the results of this trial are not yet available, certain conclusions can be reached. Preliminary examination of the data demonstrates that patients are reporting pain relief after each treatment with an ENM device. However, a more significant and outstanding observation is that during a one week trial of an ENM device the overall level of pain suffered by the user decreased as time went by, in other words, by day seven the pre-treatment pain level is significantly lower than the pre-treatment pain level earlier in the week.

Throughout this document, the term patient is not limited to humans, but can be understood as relating to any vertebrate species including mammals. This can include animals such as cats, dogs and horses.

Whilst the preferred embodiment has been described as being powered by a battery, it will be appreciated that any power source could be utilised to power the device, including a power supply comprising a transformer, and suitable for connection to a mains electricity supply.

Where upper and/or lower limits are mentioned alone or in combination, these can be combined with other lower and/or upper limits even if not expressly mentioned herein, to the extent that such is logically consistent.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An apparatus for applying electrical pulses to a patient's body by at least two electrodes at respective locations on the patient's body, the apparatus comprising a pulse generating unit connectable to the electrodes, the pulse generating unit being arranged to provide a series of electrical pulses, wherein said series of pulses comprises a plurality of first and second polarity impulses having a temporal spacing between the first and second impulses, wherein each impulse has a width of between 2 to 30 μS.

2. An apparatus as claimed in claim 1, wherein the first polarity is positive or negative and the second polarity is of the opposite polarity to the first polarity.

3-44. (canceled)

45. An apparatus as claimed in claim 1, wherein each impulse has a width of more than 10 μS or wherein each impulse has a width of 15 to 20 μS.

46. An apparatus as claimed in claim 1, wherein said series of pulses has a spacing of at least 4 μS between impulses or wherein said series of pulses has a spacing of at least 6 μS between impulses or wherein said series of pulses has a spacing of at least 10 μS between impulses.

47. An apparatus as claimed in claim 1, wherein said series of pulses has a spacing of at least 20 μS between impulses.

48. An apparatus as claimed in claim 1, having a maximum spacing of 10 μS between impulses or a maximum spacing of 20 μS between impulses.

49. An apparatus as claimed in claim 1, wherein a temporal space exists between a plurality of contiguous impulses.

50. An apparatus as claimed in claim 1, wherein a temporal space exists between a majority of impulses.

51. An apparatus as claimed in claim 1, wherein a temporal space exists between all impulses.

52. An apparatus as claimed in claim 1, wherein each impulse has an asymmetric shape.

53. An apparatus as claimed in claim 52, wherein the transition time from 0 Volts to a peak magnitude is less than or equal to 30% of the impulse width or the transition time from 0 Volts to the peak magnitude is less than or equal to 10% of the impulse width or wherein the transition time from 0 to the peak magnitude is less than or equal to 5% of the impulse width or wherein the transition time from 0 to the peak magnitude is less than or equal to 1% of the impulse width.

54. An apparatus as claimed in claim 1, wherein the transition time between the positive voltage peak and the negative voltage peak is at least 70% of the pulse period.

55. An apparatus as claimed in claim 1, wherein said impulses have a peak amplitude lying within the range 50 to 450 Volts, plus or minus respectively.

56. An apparatus as claimed in claim 55, wherein each impulse has an amplitude within the range 150 to 250 Volts, plus or minus respectively.

57. An apparatus as claimed in claim 1, wherein the magnitude of positive peak amplitude is substantially equal to the magnitude of the negative peak amplitude.

58. An apparatus as claimed in claim 1, wherein during the spacing between impulses the output of the pulse generating unit remains at a level substantially equal to zero Volts.

59. An apparatus as claimed in claim 1, wherein the series of impulses are delivered at a predetermined frequency lying within the range 100 Hz to 250 kHz or wherein the series of impulses are delivered at a predetermined frequency lying within the range 1 kHz to 250 kHz or wherein the predetermined frequency lies within the range 1 kHz to 5 kHz or the predetermined frequency lies in the range 2 kHz to 3 kHz.

60. An apparatus as claimed in claim 1, in which said series of pulses comprise a third impulse spaced from the second impulse by a temporal spacing.

61. An apparatus as claimed in claim 1, wherein the series of impulses is an intermittent series of pulses.

62. An apparatus as claimed in claim 61, wherein, in said intermittent series of electrical impulses, the ratio of the time period for which no impulses are being provided to the time period for which impulses are being regularly provided is within the range 1:3 to 1:20 and preferably 1:10.

63. An apparatus as claimed in claim 61, wherein at least one pause occurs in said intermittent series of impulses at least once every second or wherein said pause is of duration of at least 0.5 millisecond.

64. An apparatus as claimed in claim 1, further comprising at least two electrodes arranged for connection to said generating unit, for supplying electrical pulses to respective locations on the patients body.

65. An apparatus according to claim 1 for providing therapy to a patient.

66. An apparatus as claimed in claim 1, wherein said apparatus is for supplying electrical pulses to two or more locations on the patients body overlying the central nervous system, such that the pulses induce analgesic effects in the central nervous system, whilst stimulating peripheral nerves that lie between the electrodes and the central nervous system to a lesser extent or not at all.

67. An apparatus as claimed in claim 1, wherein said apparatus is for providing iontophoresis to a patients body by at least two iontophoresis electrodes at respective locations on the patient's body, the apparatus comprising a pulse generating unit connectable to the electrodes, the pulse generating unit being arranged to provide a series of electrical pulses having a peak amplitude of at least 50 Volts.

68. An apparatus as claimed in claim 67, further comprising at least two iontophoresis electrodes arranged for connection to said generating unit, for supplying electrical pulses to respective locations on the patient's body, at least one of said electrodes incorporating a medication in ionic form for application to the patient's body.

69. A method for applying electrical pulses to a patients body by utilising at least two electrodes at respective locations on the patients body, the method comprising applying an intermittent series of electrical pulses.

70. A method for providing iontophoresis to a patient by utilising at least two electrodes at respective locations on the patients body, at least one of the electrodes incorporating an ionic medication, the method comprising applying a series of pulses, each pulse having a peak amplitude of at least 50 Volts to the electrodes, such that the medication is passed into the body of the patient.
